(19) United States
(12) Patent Application Publication
(10) Pub. No.: US 2011/0171199 A1
(43) Pub. Date: Jul. 14, 2011

(54) METHOD FOR REDUCING OR ELIMINATING PAIN ASSOCIATED WITH A POST-OPERATIVE WOUND

(76) Inventor: David Tak Wai Chiu, Bronxville, NY (US)

(21) Appl. No.: 12/686,284

(22) Filed: Jan. 12, 2010

(51) Int. Cl. A61K 38/48; A61P 25/00

(52) U.S. Cl. 424/94.64

(57) ABSTRACT

The present invention relates to methods for ameliorating or preventing pain associated with a post-operative wound and kits related thereto. The method comprises administering to the wound an effective amount of an anti-clotting agent solution and administration of an effective amount of an analgesic mixture solution comprising a fast-acting analgesic and a slower-acting analgesic.

### FIELD OF THE INVENTION

The present invention relates to a method for ameliorating or preventing pain associated with a post-operative wound. The method comprises administering to the wound an effective amount of an anti-clotting agent solution and an effective amount of an analgesic mixture solution comprising a faster-acting analgesic and a slower-acting analgesic. The invention also relates to a kit embodied by the invention.

### BACKGROUND

Post-operative pain is a concern of the potential surgical patient. Therefore, it is useful to create new a method for ameliorating or eliminating post-operative pain. The method could also be employed to ameliorate or eliminate the pain associated with any open wound.

### SUMMARY OF THE INVENTION

In one aspect, the invention is method for the reduction or elimination of pain caused by a post-operative wound. The method comprises administering an effective amount of an anti-clotting agent solution to the wound and an effective amount of an analgesic mixture solution comprising a faster-acting analgesic and a slower-acting analgesic to the wound, wherein the pain is reduced or eliminated. The anti-clotting agent and analgesic mixture may be administered simultaneously or consecutively. Optionally, a vasoconstrictive agent may also be administered. The components are typically administered to a wound by applying an even spray of each solution.

In a second aspect, the invention is a kit for implementing the reduction or elimination of pain caused by a post-operative wound or open wound. The kit comprises an effective amount of an anti-clotting agent for preparing an anti-clotting solution and an effective amount of a faster-acting analgesic and a slower-acting analgesic for preparing an analgesic mixture solution. Furthermore, the kit may include an effective amount of a vasoconstrictive drug. The kit includes a spray device for administering the prepared solutions on a wound.

### DETAILED DESCRIPTION OF THE INVENTION

The present method reduces or eliminates pain associated with an post-operative wound. The post-operative wound may be associated with any type of surgery including rhytidectomy, carpal tunnel release, and parotidectomy. The method of the invention may also be useful for reducing or eliminating pain associated with any open wound.

In one embodiment of the method, an effective amount of an anti-clotting agent solution is administered to the wound followed by administration of an effective amount of an analgesic mixture solution comprising a faster-acting analgesic and a slower-acting analgesic to the wound.

The clotting agent to be administered may be homologous, heterogenous or autogenous. One example of such a clotting agent is human thrombin, human recombinant thrombin or bovine thrombin.

The faster-acting analgesic is a fast-acting, hydrophilic and topically active analgesic. Typically, the fast-acting analgesic will start reducing pain within 2 minutes with a half-life of under 2 hours. Examples of such faster-acting analgesic include lidocaine, xylocaine, tetracaine, cocaine, dibucaine, benzocaine mepivacaine, and procaine.

The slower-acting analgesic is also hydrophilic, and long acting. Typically, the slower-acting analgesic will start reducing pain within 45 minutes. Examples of such slower-acting analgesic include bupivacaine, etidocaine, prilocaine ropivacaine, and levobupivacaine.

The faster-acting analgesic and the slower-acting analgesic are typically administered in combination. However, the may be administered separately if desired. The clotting agent activates clotting and minimizes oozing. The faster-acting analgesic and slower-acting analgesic form part of a complex clot. This clot will then be metabolized releasing the faster-acting and slower-acting analgesics over time. A thrombin clot is normally metabolized within 12 hours.

In a second embodiment of the method, an effective amount of an anti-clotting agent solution is administered to the wound in combination with an effective amount of an analgesic mixture solution comprising a faster-acting analgesic and a slower-acting analgesic to the wound.

The clotting agent to be administered may be homologous, heterogenous or autogenous. One example of such a clotting agent is human thrombin, human recombinant thrombin or bovine thrombin.

The faster-acting analgesic is a fast-acting, hydrophilic and topically active analgesic. Examples of such fast-acting analgesic include lidocaine, xylocaine, tetracaine, cocaine, dibucaine, benzocaine mepivacaine, procaine.

The slower-acting analgesic is also hydrophilic, and long acting. Typically, the slower-acting analgesic will start reducing pain within 45 minutes. Examples of such slower-acting analgesic includes bupivacaine, etidocaine, prilocaine ropivacaine, and levobupivacaine.

The clotting agent activates clotting and minimizes oozing. The fast-acting analgesic and slower-acting analgesic form part of a complex clot. This clot will then be metabolized releasing the fast acting and slow acting analgesics over time.

The method may further comprise the administration of a vasoconstrictive agent including epinephrine, norepinephrine, the catecholamines, epinephrine isoproterenol, dopamine, ephedrine, phenylephrine, amphetamine, metraminol, methoxamine, ergot alkaloids, ergonovine, methylergonavine, methysergide, ergotamine, an angiotensin, and a prostaglandin.

The compounds described above may be administered in a spray or a gel to obtain the benefits of the invention. When thrombin is administered in a spray typical concentrations of thrombin will be at between 1000 to about 2000 units/ml, for example 1000, 1100, 1200, 1300, 1400, 1500, 1600, 1700, 1800, 1900 or 2000 units/ml. A typical amount of lidocaine administered is between 5 to 300 mg, preferably between 50 and 200 mg, for example 20 mg, 40 mg, 60 mg, 80 mg, 100 mg, 120 mg, 140 mg, 160 mg, 180 mg, 200 mg, 220 mg, 240 mg, 260 mg, 280 mg, or 300 mg, not to exceed 4.5 mg/kg, for example 1 mg/kg, 2 mg/kg, 3 mg/kg or 4 mg/kg. A typical amount of bupivacaine administered is between 20 to 225 mg, preferably between 25 and 100 mg, for example 50 mg, 75 mg, 100 mg, 125 mg, 150 mg, 175 mg, 200 mg, or 225 mg. Similar physiologically effective amounts can be used when other faster-acting and slower-acting analgesics, when administered are employed. When epinephrine is optionally administered it is administered at concentrations of between 0.01-0.02 mg/ml.

In a further embodiment, the invention is a kit. The kit will provide all the required ingredients in dry form which can be solubilized to an appropriate concentration prior to administration. In one embodiment the kit will comprise an anti-clotting agent, a slower-acting analgesic, and a faster-acting analgesic, a spray device, and, optionally, a vasoconstrictor agent.

It will be apparent to those skilled in the art that many modifications, variations and alterations to the present disclosure, both to materials and methods, may be practiced. Such modifications, variations and alterations are intended to be within the spirit and scope of the present invention.

The practice of the invention is illustrated by the following non-limiting examples.

### EXAMPLE

A. Thrombin solution: A solution of thrombin, either allogenic or heterogenic, was reconstituted with sterile isotonic saline at a concentration of 1,000 to 2,000 units/ml by sterile syringe or sterile transfer needle.

B. 2% Lidocaine HCl solution: each ml contained lidocaine HCl 20 mg, sodium chloride 6 mg, methylparaben 1 mg. The pH was adjusted to 6.5.

C. 0.5% bupivacaine solution: each ml contained bupivacaine 5 mg, methyparaben 1 mg, sodium chloride to adjust to istonic and NAOH or HCL to adjust pH to 6.5

At the conclusion of surgery and after active bleeding was under control, the wound was sprayed first with the solution of thrombin followed by a mixture of lidocaine/bupivacaine. Typically about 5 mls of the solutions were employed. The applicator was designed to give an even spray of each solution. Application of the combination of thrombin, lidocaine and bupivacaine will bring about immediate analgesia as well as a prolonged analgesic state which lasts for 12 to 24 hours.

Although specific methods and kits have been described, it will be appreciated that various modifications and changes may be made therein without departing from the spirit and scope of the invention as defined by the following claims:

I claim:

1. A method for the reduction or elimination of pain caused by a post-operative wound, said method comprising first administering an effective amount of an anti-clotting agent solution to the wound followed by administering an effective amount of an analgesic mixture solution comprising a faster-acting analgesic and a slower-acting analgesic to the wound, wherein the pain is reduced or eliminated.

2. The method of claim 1, wherein said analgesic mixture solution further comprises a vasoconstrictive drug in an effective amount.

3. The method of claim 1, wherein said administering is designed to give an even spray of each solution.

4. The method of claim 1, wherein said anti-clotting agent is thrombin.

5. The method of claim 1, wherein the faster-acting analgesic is lidocaine.

6. The method of claim 1, wherein the slower-acting analgesic is bupivacine or levobupivacine.

7. The method of claim 2, wherein the vasoconstrictive drug is epinephrine.

8. A kit for the reduction or elimination of pain caused by a post-operative wound, said kit comprising an effective amount of an anti-clotting agent for preparing an anti-clotting agent solution and effective amount of a faster-acting analgesic and a slower-acting analgesic for preparing an analgesic mixture solution.

9. The kit of claim 8, wherein said kit further comprises a vasoconstrictive drug in an effective amount.

10. The kit of claim 8, wherein said kit further comprises a spray device.

11. The kit of claim 8, wherein said anti-clotting agent is thrombin.

12. The kit of claim 8, wherein the faster-acting analgesic is lidocaine.

13. The kit of claim 8, wherein the slower-acting analgesic is bupivacaine or levobupivacine.

14. The kit of claim 9, wherein the vasoconstrictive drug is epinephrine.

15. A method for the reduction or elimination of pain caused by a post-operative wound, said method comprising administering an effective amount of anti-clotting agent solution to the wound in combination with an effective amount of an analgesic mixture solution comprising a faster-acting analgesic and a slower-acting analgesic to the wound, wherein the pain is reduced or eliminated.

16. The method of claim 15, wherein said analgesic mixture solution further comprises a vasoconstrictive drug in an effective amount.

17. The method of claim 15, wherein said administering is designed to give an even spray of each solution.

18. The method of claim 15, wherein said anti-clotting agent is thrombin.

19. The method of claim 15, wherein the faster-acting analgesic is lidocaine.

20. The method of claim 15, wherein the slow-acting analgesic is bupivacaine or levobupivacaine.

21. The method of claim 16, wherein the vasoconstrictive drug is epinephrine.
